(19) United States
(12) Patent Application Publication
(10) Pub. No.: US 2025/0174111 A1
(43) Pub. Date: May 29, 2025

(54) Non-Dispersive and Proximity Sensors

(71) Applicant: Focus Universal Inc., Ontario, CA (US)

(72) Inventor: Desheng Wang, Diamond Bar, CA (US)

(21) Appl. No.: 19/039,007

(22) Filed: Jan. 28, 2025

Related U.S. Application Data
(63) Continuation-in-part of application No. 18/050,803, filed on Oct. 28, 2022.
Application No. 18/050,803 is a continuation of application No. 16/777,632, filed on Jan. 30, 2020, now Pat. No. 11,488,468.
(63) Continuation-in-part of application No. 18/421,258, filed on Jan. 24, 2024.
Application No. 18/421,258 is a continuation of application No. 17/749,982, filed on May 20, 2022, now Pat. No. 11,913,879.
(60) Provisional application No. 63/190,934, filed on May 20, 2021.

(51) Int. Cl. G08B 25/10; G08B 3/10; H04W 4/80

(52) U.S. Cl. CPC G08B 25/10; G08B 3/10; H04W 4/80

(57) ABSTRACT

Disclosed is a sensor for detecting the proximity of smart devices previously connected to the sensor using the IEEE 802.11 protocols. Importantly, the sensor does not operate using a Bluetooth® connection, which would require the smart devices previously connected to the sensor using the IEEE 802.11 protocols to also have Bluetooth® protocols enabled. Enabling such Bluetooth® protocols uses more battery than having the IEEE 802.11 protocol adapter enabled. The sensor references a list of previously connected smart devices and attempts to connect with any in range, if none can be connected to, the sensor emits an audible alarm signal. Further, no application is required for the sensor to operate.

### CROSS-REFERENCE TO RELATED APPLICATIONS

The present application is a continuation-in-part of U.S. patent application Ser. No. 18/050,803 (FCUV-114-CON), filed Oct. 28, 2022 and titled “SENSOR FOR DETECTING THE PROXIMITY OF AN IEEE 802.11 PROTOCOL CONNECTABLE DEVICE”, which is a continuation of U.S. patent application Ser. No. 16/777,632, filed Jan. 30, 2020.

The present application is a continuation-in-part of U.S. patent application Ser. No. 18/421,258 (FCUV-119-CON), filed Jan. 24, 2024 and titled “NON-DISPERSIVE INFRARED SENSOR”, which is a continuation of U.S. patent application Ser. No. 17/749,982, filed May 20, 2022, which claims the benefit of priority to U.S. Provisional Patent Application No. 63/190,934, filed May 20, 2021.

### FIELD

The present disclosure relates to relates to a device for detecting the proximity of an IEEE 802.11 protocol connectable device, as well as sensors which detect the presence of a gas.

### BACKGROUND

Use of mobile devices which typically connect to local area networks using a IEEE 802.11 protocol are a constant for many users in every day life. The most prevalent may be the smart phone. While most users have a smart phone for making calls, they typically use these devices as small computers as well. In order to get more reliable data connections to the internet, users often prefer to connect to a local area network and out through the local area network to the internet, rather than using less reliable and potentially lower bandwidth cellular connections. For this reason, most users leave their WiFi® service, which used the IEEE 802.11 protocol, on their smartphone active.

However, because users tend to turn to their smart phones for a myriad of tasks, they often set their smart phone down where they last used it. This can mean more easily forgetting their smart phone. A user may get in a car and leave their phone behind. When this happens at home it's an inconvenience, when this happens at a commercial establishment, it may have more serious consequences.

A common denominator with both leaving the house and a commercial location in many areas is an automobile. Almost all automobiles have a power source accessible to a user, in fact, many users purchase and user power adapters to charge their phone while in the car.

For the foregoing reasons, there is a need for a device which can notify a user that their smart device is not in the car with them.

In state-of-the-art non-dispersive infrared (NDIR) sensors, an infrared (IR) lamp directs waves of light through a tube filled with a sample of air toward an optical filter in front of an IR light detector. The IR light detector measures the amount of IR light that passes through the optical filter. The remainder of the IR light has been absorbed by the gas in the sample. One example of a NDIR sensor is a NDIR sensor to detect the presence of carbon dioxide (CO2). The band centered on a 4.2 micron wavelength of IR radiation produced by a lamp in a CO2 NDIR sensor is very close to the 4.26 micron absorption band of CO2. Because the IR spectrum of CO2 is unique, matching the light source wavelength serves as a signature or “fingerprint” to identify the CO2 molecule.

The IR lamp produces light which passes through a length of the tube, the CO2 gas molecules absorb the specific band of IR light while letting other wavelengths of light pass through. At the detector end the remaining light hits an optical filter that absorbs every wavelength of light except the 4.2 micron wavelength absorbed by CO2 molecules in the air sample tube. Finally, an IR detector reads the remaining amount of 4.2 micron light that was not absorbed by the CO2 molecules or the optical filter.

The difference between the amount of light radiated by the IR lamp and the amount of IR light received by the detector is measured. Since the difference is the result of the light being absorbed by the CO2 molecules in the air inside the tube, it is directly proportional to the number of CO2 molecules in the air sample tube. All measurements start out as analog micro-voltages. While some sensors output this as an analog voltage or 4-20 mA signal, some also include an analog to digital converter on the sensor PCB that converts the voltages into serial or RS-485 output. Serial output is especially useful for using NDIR CO2 sensors with Arduino or Raspberry Pi micro controllers.

A continuing problem in state-of-the-art NDIR sensors has been size. As discussed in the example above, there must be a tube to capture the sample. The necessity of this tube to capture the sample and contain the IR spectrum light in combination with the fact that a minimum length is necessary to get a quality detection means that it is difficult to reduce the size of the sensor below the optimum length for the tube, causing a size/performance tradeoff. Thus, the optimum sensor will have a long tube length essentially precluding a small overall sensor size.

Another problem with state-of-the-art NDIR sensors is that the IR lamp heats the sample tube. In addition to the obvious problem of heat itself impacting the operation of the circuit components on the circuit board of the NDIR sensor, the heat generated by the IP lamp located on one end of the sample tube may have other negative effects. Many NDIR sensors uses a large number of thermocouples connected either in series or parallel to create a thermopile. The output voltage of the series-connected thermocouples depends on the temperature difference between the thermocouple junctions and the reference junction. If the reference junction is heated by the lamp, there can be problems with the output voltage. This results in incorrect data fed from the detector, and can lead to false negative detections.

For the foregoing reasons, there is a need for a sensor which optimizes accuracy in detection, but also comes in a small package.

### SUMMARY

Disclosed herein is a sensor for detecting the proximity of a wireless connectable smart device using the IEEE 802.11 protocols. The sensor may include a housing. The housing may include a distal end portion and a proximal end portion. The distal end portion may be configured to be placed in an automotive cigarette lighter, and the proximal end portion may include one or more power sockets for connecting one or more charging cables. The sensor may further include interface surfaces on the distal end portion to draw power from the cigarette lighter. The sensor may further include a power supply. The power supply may be connected to the interface surfaces. The power supply may regulate the voltage of the power. The sensor may further include a transceiver using the IEEE 802.11 protocols. The transceiver may be located in the housing. The transceiver may be electrically connected to the power supply. The transceiver may be configured to connect to IEEE 802.11 protocol capable smart devices within range of the transceiver. The sensor may further include a processor. The processor may be located in the housing and electrically connected to the transceiver. The sensor may further include a memory located in the housing. The memory may be electrically connected to the processor. The memory may have instructions stored on it for execution on the processor. The sensor may have an audio circuit electrically connected to the processor. The audio circuit may include an audio driver for generating audible sounds. The instructions stored on the memory may include instructions that when the automobile is started and power is provided to the transceiver, the instructions cause the transceiver to search for, and connect to, smart devices previously connected to the sensor using the IEEE 802.11 protocols. If the transceiver fails to connect to any previously connected smart devices using the IEEE 802.11 protocols, the instructions cause the audio circuit to emit an audible alarm.

Further disclosed is a method for manufacturing a sensor for detecting the proximity of a smart device connectable to a transceiver using IEEE 802.11 protocols. The method may include forming a housing. The housing may include a distal end portion. The distal end portion may be configured to be placed in an automotive cigarette lighter, and the housing may define an interior of the housing. The method may further include adding a transceiver to the interior of the housing. The transceiver may use IEEE 802.11 protocols to connected to IEEE 802.11 protocol capable smart devices within range of the transceiver. The method may further include adding a processor to the interior of the housing. The processor may be electrically connected to the transceiver. The method may further include adding a memory to the interior of the housing. The memory may be electrically connected to the processor. The memory may have instructions stored on it for execution on the processor. The method may further include adding an audio circuit electrically connected to the processor. The audio circuit may include an audio driver for generating audible sounds. The instructions stored on the memory may include instructions that when the automobile is started and power is provided to the transceiver, the instructions may cause the transceiver to search for, and connect to, smart devices previously connected to the sensor using the IEEE 802.11 protocols. If the transceiver fails to connect to any previously connected smart devices using the IEEE 802.11 protocols, the instructions may cause the audio circuit to emit an audible alarm.

Further disclosed is a sensor for detecting the proximity of a wireless connectable smart device using the IEEE 802.11 protocols. The sensor may include a housing. The housing may include a distal end portion. The distal end portion may be configured to be placed in an automotive cigarette lighter. The housing may define an interior of the housing. The sensor may further include interface surfaces on the distal end portion. The interface surfaces may draw power from the cigarette lighter. The sensor may further include a transceiver which may use the IEEE 802.11 protocols, The transceiver may be located in the interior of the housing and may be electrically connected to the interface surfaces. The transceiver may be configured to connect to IEEE 802.11 protocol capable smart devices within range of the transceiver. The sensor may further include a processor. The processor may be located in the housing and may be electrically connected to the transceiver. The sensor may further include a memory. The memory may be located in the housing and may be electrically connected to the processor. The memory may have instructions stored on it for execution on the processor. The sensor may further have an audio circuit, which may be electrically connected to the processor. The audio circuit may include an audio driver for generating audible sounds. The instructions stored on the memory may include instructions that when the automobile is started and power is provided to the transceiver, the instructions may cause the transceiver to search for, and connect to, smart devices previously connected to the sensor using the IEEE 802.11 protocols. If the transceiver fails to connect to any previously connected smart devices using the IEEE 802.11 protocols, the instructions may cause the audio circuit to emit an audible alarm.

In some embodiments, a sensor for detecting the presence of one or more gasses includes an IR lamp that generates light in the IR spectrum. The sensor includes a first light pipe section having a first end portion and a second end portion. The first end portion abuts the IR lamp and the first light pipe section passes the light through from the first end portion to the second end portion. The sensor includes a first sample tube section receiving light from the first light pipe section. The first sample tube section has a first interior containing a first sample. The first sample includes a mix of gases from the local atmosphere. The IR spectrum light passes through the interior of the first sample tube section to a second end portion of the first sample tube section. The sensor includes a second light pipe section that has an arcuate shape so that a first end and a second end of the second light pipe section face the same direction. The first end portion of the second light pipe section can be located adjacent to a second end portion of the first sample tube section. The second light pipe section can receive the light at the second end portion of the first sample tube section and transmit the light from the first end portion to a second end portion. The sensor can include a second sample tube section with a first end portion and a second end portion. The first end portion of the second sample tube section can be located adjacent to the second end portion of the second light pipe section. The first end portion of the second sample tube section can receive the light from the second end of the second light pipe section. The second sample tube section can have a second interior containing a second sample. The second sample can include the mix of gases from the local atmosphere, such that the light passes through the interior of the second sample tube section to a second end portion of the second sample tube section. The sensor can include a first filter located at the second end portion of the second sample tube section. The first filter can bandpass a band of IR spectrum light centered on a wavelength that is indicative of the presence of a gas in the mix of gases from the local atmosphere. The sensor can include a first detector located behind to the first filter. The detector can be configured to detect the amount of IR spectrum light in the band of IR spectrum light.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 shows a schematic diagram of a portion of the sensor.

FIG. 2 shows a view of the interior of the first sample tube section and the second light pipe section.

FIG. 3 shows a view of the interior of the second sample tube section and a plurality of decoder windows covered by filters.

FIG. 4 shows a graph of some gasses at their peak absorption wavelengths.

FIG. 5 shows a perspective view of one embodiment of the sensor.

FIG. 6 shows a schematic diagram of an exemplary sensor.

### DETAILED DESCRIPTION

The detailed description set forth below in connection with the appended drawings is intended as a description of the presently preferred embodiment of a sensor for detecting various gasses which may be present in a local atmosphere, and is not intended to represent the only form in which it can be developed or utilized. The description sets forth the functions for developing and operating the system in connection with the illustrated embodiments. It is to be understood, however, that the same or equivalent functions may be accomplished by different embodiments that are also intended to be encompassed within the scope of the present disclosure. It is further understood that the use of relational terms such as first, second, distal, proximal, and the like are used solely to distinguish one from another entity without necessarily requiring or implying any actual such relationship or order between such entities.

The detailed description set forth below in connection with the appended drawings is intended as a description of the presently preferred embodiment of a device to notify a user when an IEEE 802.11 protocol connectable smart device is not in an automobile, and is not intended to represent the only form in which it can be developed or utilized. The description sets forth the functions for developing and operating the system in connection with the illustrated embodiments. It is to be understood, however, that the same or equivalent functions may be accomplished by different embodiments that are also intended to be encompassed within the scope of the present disclosure. It is further understood that the use of relational terms such as first, second, distal, proximal, and the like are used solely to distinguish one from another entity without necessarily requiring or implying any actual such relationship or order between such entities.

Disclosed is a sensor for detecting the proximity of a wireless connectable smart device using the IEEE 802.11 protocols, such as those used for WiFi® communication. The sensor only uses the IEEE 802.11 protocols in order to achieve two important ends. First, the IEEE 802.11 protocols offer a reliable way to search for, and connect to, devices in range which also use the IEEE 802.11 protocols. As used herein, the term smart device is meant to include any device which uses the IEEE 802.11 protocols to connect to another device.

Typically, users with smart devices with such a capability leave this capability turned on. This fact points to the second important end. That is, other prior art sensors typically operate using Bluetooth® protocols. However, operating using Bluetooth® protocols is only effective if the smart device is Bluetooth® protocol capable, and has the Bluetooth® capability turned on. Activating Bluetooth® capability typically results in a significant battery drain of the smart device. In order to conserve battery power, many users do not activate Bluetooth® connectivity on their smart devices. If this connectivity is not activated on the smart device, it renders the sensor useless. Thus, the disclosed sensor operates using IEEE 802.11 protocols.

### Example Non-Dispersive Infrared Sensor

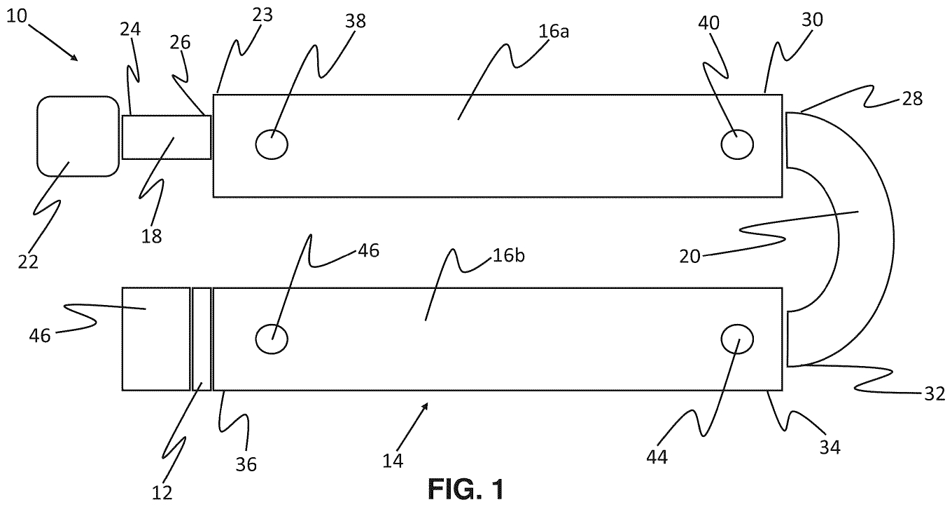

FIG. 1 illustrates an overview of a portion of the gas detection sensor 10 according to the present invention. The gas detection sensor 10 may be used to detect a number of different gasses depending on the filter 12 used by the sensor.

The gas detection sensor 10 may include a plurality of components. Collectively, the components may allow the sensor 10 to generate light in the IR spectrum, guide the emission of the IR spectrum light through a sample, and then filter and detect the emission after passing through the sample.

An IR lamp 22 may be the source of the IR emission. The IR lamp 22 may be located on a first end portion 24 of a first section 18 of light pipe. The closer the light pipe first section 18 is placed to the IR lamp 22, the more efficiently the light pipe will operate. In preferred embodiments, the first section 18 of the light pipe abuts the IR lamp 22. A light pipe body extends between the first end portion 24 and a second end portion 26 of the first section 18. They body may have a cylindrical cross section. Alternatively, the body may have a square cross section, a rectangular cross section, an octagonal cross section or any cross section which allows for the efficient transmission of the IR source light.

The gas detection sensor 10 may include a sample tube 14 of two sections 16a, 16b. The second end portion 26 of the first section 18 of light pipe may be located substantially at a first end portion 23 of a first section 16a of the sample tube. Thus, the first section 18 of light pipe may be located entirely before the first section of sample tube 16a. Alternatively, a second end portion 26 may be partially located within the first section 16a of the sample tube.

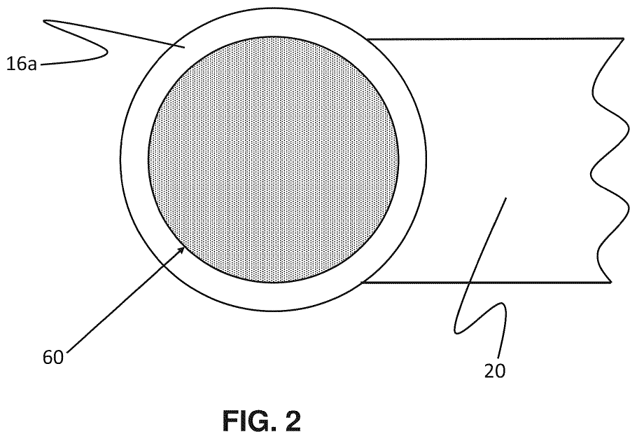

As shown in FIGS. 1 and 2, the first sample tube section 16a may be a hollow tube. That is, the first section 16a may have a first end portion 23. The first end portion 23 may include the face of a cylindrical wall forming a body of the tube. The face of the cylindrical wall may define a circular first end opening. The cylindrical wall may also define an interior 60 of first section 16a of the sample tube. The first sample tube section 16a may include a first aperture 38 which allows gas to enter the interior of the first sample tube section 16a. The first aperture 38 may be located on the first end portion 23 of the first section 16a. The first aperture 38 may be an opening in the cylindrical wall of the first section 16a. The first section may further have a second aperture 40 which allows gas to pass from the interior of the first section 16a to an exterior of the first section 16a. The second aperture 40 may be located near a second end portion 30 of the first section 16a. Similar to the first aperture 38, the second aperture 40 may be an opening in the cylindrical wall of the first section 16a. That is, a portion of the material of the cylindrical wall may be removed to place the interior of the first section 16a in fluid communication with the exterior of the first section 16a.

As shown in FIGS. 1 and 2, a second light pipe section 20 may be located between the first sample tube section 16a and second sample tube section 16b. The second light pipe section 20 may be curved. The second light pipe section 20 may be made from a rigid material. Alternatively, the second light pipe section 20 may be made from a flexible material. A first end portion 28 of the curved second light pipe section 20 may be substantially located near or overlapping a second end portion 30 of the first sample tube section 16a. A second end portion 32 of the curved second light pipe section 20 may be substantially located at a first end portion 34 of the second sample tube section 16b. As with the first light pipe section 18, end portions 28, 32 of the second light pipe section 20 may be located partially within the sample tube sections 16a, 16b. An outer circumference of the second light pipe section 20 may be substantially equal to an inner diameter of the cylindrical wall of the first sample tube section 16a.

The second light pipe section 20 may be rigid or flexible. If it is rigid it may be formed in a ‘u’ shape of predetermined dimensions. If the second light pipe section 20 is flexible, the section may be cut to length, and adapters attached to connect the flexible light pipe securely to the second end portion 30 of the first sample tube section 16a, and the first end portion 34 of the second sample tube section 16b.

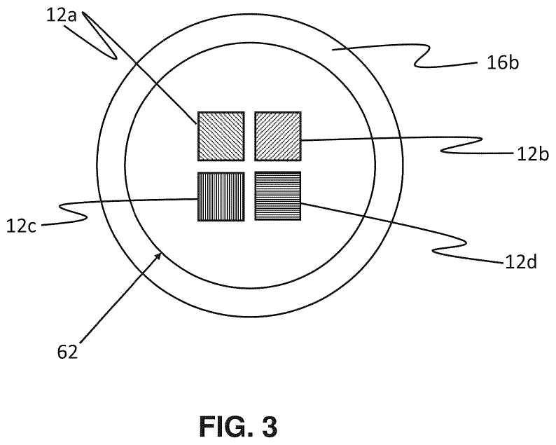

As shown in FIGS. 1 and 3, the second sample tube section 16b may be similar to the first sample tube section 16a. The second light pipe section 20 directs the light from the second end portion 30 of the first sample tube section 16a to the first end portion 34 of the second sample tube section 16b. The second sample tube section 16b may include a hollow tube. The first end portion 34 of the second sample tube section 16b may include the face of a cylindrical wall forming body of the tube. The face of the cylindrical wall may define a circular first end opening. The cylindrical wall may also define an interior 62 of first sample tube section 16a. The second sample tube section 16b may include a first aperture 44 which allows gas to enter the interior of the sample tube. The first aperture 44 may be located on the first end portion 34 of the second sample tube section 16b. The first aperture 38 may be an opening in the cylindrical wall of the second sample tube section 16b. The second sample tube section 16b may further have a second aperture 46 which allows gas to pass from the interior 62 of the second sample tube section 16b to an exterior of the second sample tube section 16b and back in to atmosphere. The second aperture 46 may be located near a second end portion 36 of the second sample tube section 16b. Similar to the first aperture 44, the second aperture 46 may be an opening in the cylindrical wall of the second sample tube section 16b. That is, a portion of the material of the cylindrical wall may be removed to place the interior of the second sample tube section 16b in fluid communication with the local atmosphere exterior to the second sample tube section 16b.

The filter 12 may be located at or overlapping a second end portion 36 of the second sample tube section 16b. The filter 12 may abut the second end portion 36 of the second sample tube section 16b in order to filter most effectively. The IR detector 42 may be located behind the filter 12. The IR detector 42 may abut the filter in order to most effectively detect the amount of light not absorbed by the sample.

The filter 12 may filter out a narrow band spectral region which overlaps with the absorption region of the gas of interest. The filter 12 may be interchangeable. For example, the filter 12 may be placed in a frame which secures the filter in an optimum location. Based on the filter, the sensor 10 may detected the presence of a particular gas. If the filter were changed, a different gas may be detected, the filter will bandpass a band centered on a different wavelength. The wavelength will correspond to the peak IR absorption wavelength of the gas to be detected, and thus may be termed a detection wavelength.

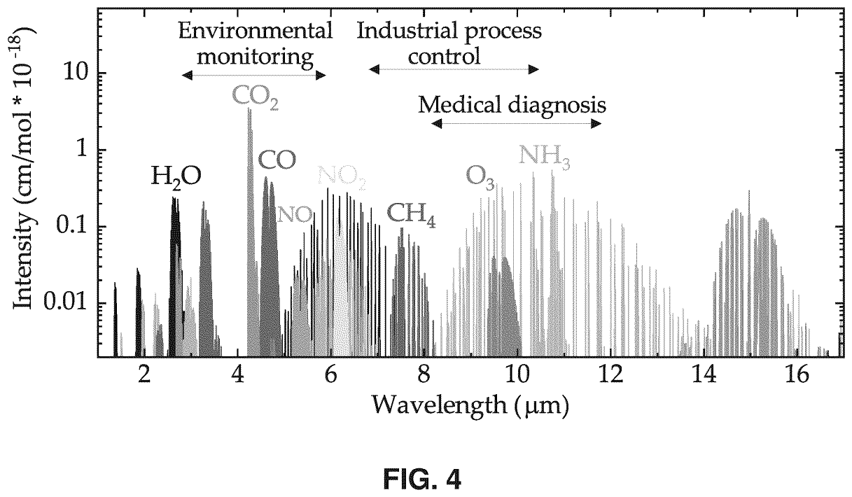

FIG. 4 provides a depiction of some gasses at their peak absorption wavelengths. FIG. 4 further provides typical uses for detection of different gasses. The detector 42 may be a single or multi-channel infrared detector. The multi-channel infrared detectors may be two channel, four channel, or eight channel detectors. In a multi-channel detector, the filter 12a-d may be integrated with the detector. Each channel on the detector is capable of detecting a different wavelength in the IR spectrum. Thus, a single detector 42 may be able to detect multiple gasses without the need to change the filter.

The multi-channel detectors may be a pyroelectric detector. Pyroelectric crystals have a rare asymmetry due to their single polar axis. This causes their polarization to change with temperature. The pyroelectric effect used in sensor technology involves a thin pyroelectric crystal coated perpendicular to the polar axis with electrodes. On the upper electrode of the crystal, an absorbing layer, also termed a black layer, may be applied. When the absorbing layer interacts with infrared radiation, the pyroelectric layer heats up and a surface charge is formed. If the radiation is switched off, a charge of the opposite polarity forms. However, the level of the charge may be very low. Before the finite internal resistance of the crystal can equalize the charges, extremely low-noise and low leakage current field-effect transistors (JFET) or one or more operational amplifiers (OpAmps) may convert the charges into a signal voltage.

The above arrangement may be placed on a first circuit board. Other circuitry, including the JFETs or OpAmps, and a second sample tube containing a reference gas, may be placed on a piggybacked circuit board. This design choice increases the overall depth of the sensor package, but does not increase the length or width, thereby maintaining the footprint size of the sensor 10.

More specifically, the IR lamp may be moved back from the state-of-the-art placement at the first end portion of the sample tube. The IR lamp 22 of the present disclosure is moved away from the first section 16a of the sample tube in order to reduce the effects of heat generated by the IR lamp 22 on the first sample tube section 16a. In addition to moving the IR lamp 22 away from the first end portion 23 of the first sample tube section 16a, the IR lamp 22 may be connected to a heat sink which takes any excess heat generated by the IR lamp 22 and directs it away from the first sample tube section 16a. The first light pipe section 18 may direct the light of the IR lamp 22 toward the first sample tube section 16a. The light pipe material is known to transmit up to 90% of the light through the length of the light pipe material.

The above arrangement of component parts of the sensor 10 achieves the important result of reducing the transmission of heat from the IR lamp to the sample tube, which is an existing problem in the state-of-the-art. The heat generated by the IR lamp can heat the sample tube, and effect the operation of the thermopile in the sensor. Because the IR lamp 22 is physically spaced apart from the first sample tube section 16a, the heat is dissipated from the IR lamp 22, either to the surrounding air or to the heat sink before it reaches the first sample tube section 16a. The dissipation of heat before the heat reaches the first sample tube section 16a prevents negative effects to the thermopile.

In some embodiments, the heat dissipation feature may alone be used. That is, the IR lamp 22 may be moved from a first end portion of a sample tube, and a light pipe used to direct the light from the IR lamp in to the sample tube. Only a single section of sample tube may be used, because the first light pipe section is all that is required to decouple the heat of the IR lamp 22 from the sample tube. Thus, these embodiments do not have a second light pipe section or a second sample tube section.

However, other embodiments do include the second light pipe section 20 and the second sample tube section 16b. This is because the gas detection sensor 10 uses a principle known either as the Beer-Lambert Law. For purposes of this disclosure, the Beer-Lambert Law is given as:

I=Iₒe^{-KLC}

Where I is the intensity of light at the detector, Io is the intensity of the light entering the sample, K is the molar absorptivity, L is the distance the light travels through the sample, and C is the concentration of the gas being tested for. Thus, the absorbance of the light is proportional to the distance the light travels through the sample. We can assume equal concentration because the gas is being tested for in the atmosphere and entropy is in effect, dispersing the tested for gas in an equal concentration, at least locally, in the atmosphere. Thus, there will be more absorption of the light with a longer sample tube. However, a long sample tube makes the sensor physically larger, and the state-of-the-art sensors are already almost too large for their desired applications.

If the sample tube may be split in to more than one section, the two sections may be placed in parallel. The placement of the two sections in parallel keeps the footprint of the sensor the same. While this creates a longer sample tube in the same footprint, it simultaneously creates a problem. The problem is that light from the IR lamp 22 cannot, on its own, curve after exiting the first section to enter the second section.

Sample tubes are designed with two apertures. A first aperture 38 may allow atmospheric gas in to the sample tube, and a second aperture 40 may allow gas to leave the sample tube. Thus, when split, each sample tube section would need a set of apertures in order for the sample tube section to obtain a sample.

A piece of light pipe may be placed between the two sections of sample tube in order to capture the light from the first section and direct the light to the second section. Specifically, a first end portion 28 of the curved second light pipe section 20 may be substantially located abutting a second end portion 30 of the first sample tube section 16a. A second end portion 32 of the curved second light pipe section 20 may be substantially located at a first end 34 of the second sample tube section 16b. Light pipe sections may be formed either straight or curved, rigid or flexible. This disclosure contemplates both rigid and flexible light pipes to direct the light from the second end portion 30 of a first sample tube section 16a to a first end 34 of the second sample tube section 16b.

In operation, the IR lamp 22 may be powered to emit light. The IR lamp 22 may be operated in such a way to chop or modulate the emission of light. This allows the detector to differentiate the radiation from the IR lamp 22 from the IR radiation present in the spectrum of atmospheric light. The light radiation from the IR lamp 22 is then directed in to and through the first light pipe section 18. With rare exception, at least 80%, and as much as 90% of the IR spectrum light from the IR lamp 22 is transmitted through the first light pipe section 18.

The IR spectrum light then enters the interior of the first sample tube section 16a. While passing through the first sample tube section 16a, the IR spectrum light will encounter the sample in the interior of the first sample tube section 16a. The sample may enter the interior though the first aperture 38. Some portion of the atmosphere adjacent to the first sample tube section 16a forms the sample in the interior 60 of the first sample tube section 16a. The sample may be continuously cycling through the interior of the first sample tube section 16a by cycling through the first aperture 38 and second aperture 40, which are both fluidly connected to the local atmosphere. If any of the one or more gases are present for which the sensor 10 is detecting, some amount of the IR spectrum light is absorbed in the first sample tube section 16a by those one or more gases. The IR spectrum light eventually reaches the second end portion 30 of the first sample tube section 16a.

After reaching the second end portion 30 of the first sample tube section 16a, the IR spectrum light enters the second light pipe section 20. The IR spectrum light enters the second light pipe section 20 at a first end portion 28. The IR spectrum light then travels through the second light pipe section 20 to a second end portion 32. Again, the light pipe transmits between 80% and 90% of the light that enters. Thus, the circuity on the sensor 10, which may include one or more processors, and one or more memories will include instructions which account for this inefficiency in order to avoid false positive detections from the loss of some of the light at the observed wavelength due to light pipe transmission inefficiencies. The curvature of the light pipe material does not cause any further inefficiencies as compared to straight pieces of the light pipe material. Further, the flexible light pipe material does not cause any further inefficiencies than a rigid piece of light pipe material. Thus, the IR spectrum light which enters the second section of light pipe material travels through the light pipe and exists with a 10% to 20% loss of the IR spectrum light which entered the second light pipe section 20. At the second end portion 32 of the second light pipe section 20, the IR spectrum light enters the second sample tube section 16b.

The IR spectrum light enters the first end portion 34 of the second sample tube section 16b. At this point the intensity of the observed wavelength may have already been reduced by up to 40% due to the inefficiencies inherent in the first light pipe section 18 and second light pipe section 20. The intensity of the observed wavelength may be reduced even further than the reductions caused by the inefficiencies by the light pipe sections 18, 20 if there is a quantity of a gas which the senor is trying to detect present in the sample of the first sample tube section 16a. Again, the inefficiencies of the light pipe material are built in to the calculations performed by the system in detecting the presence of a gas, so these inefficiencies will not product false positive detections. The IR spectrum light traverses across the second section 16b of sample tube from a first end portion 34 to a second end portion 36. The filter 12 and the detector 42 may be attached to or abut the second end portion 36 of the second sample tube section 16b.

The sample in the second sample tube section 16b may be nearly identical to the sample in the first sample tube section 16a. This is because both samples are drawn from the atmosphere in the immediate vicinity of the sensor 10. Similar to the first sample tube section 16a, the sample may flow in and out through the first aperture 44 and the second aperture 46 in the second sample tube section 16b. Again, this allows continuous flow through the interior 62 the second sample tube section 16b, ensuring that if any of the one or more gasses the sensor 10 is detecting for is present in the atmosphere, the one or more gasses are also present in the sample. If any of the one or more gasses are present, then the one or more gasses may absorb light at the detection wavelength in the IR spectrum.

As shown in FIG. 3, after passing through the second sample tube section 16b, the IR spectrum light may arrive at the one or more filters 12a-d. As discussed above, the one or more filters may be a separate component, or the one or more filters may be integrated with the detector 42. Regardless of the physical location, the one or more filters 12a-d perform the same function. Specifically, as light arrives at the one or more filters 12a-d, the one or more filters 12a-d bandpass light in a narrow band centered on a detection wavelength which is absorbed by the gas the sensor is working to detect. If there is a multi-channel filter/detector combination, then each detector may have an individual filter 12a-d. For example, if the sensor 10 includes a four channel detector 42, as is shown in FIG. 3, then the detector may have a filter 12a-d for each channel. As discussed above, it is understood that the detector may be a one, two four, or eight channel detector, with a corresponding number of filters. Each filter will have different bandpass characteristics. Specifically, each filter will bandpass a band centered on a different detection wavelength. Each channel is therefore able to detect the presence of a different gas. After the IR spectrum light arrives at the detector, the light may cause an increase in current on the detector, as is described above. Through circuitry well known in the art, the signal may be amplified and converted to a voltage signal in order to determine if the detected IR spectrum light is indicative of the presence of one or more gasses.

After the detector makes the measurement of the amount of IR light at a wavelength reaching the detector, the detector passes a signal to circuitry for further processing. This circuitry is well known in the art and determines if the gas being detected is present in the sample. However, unlike state-of-the-art circuitry, the circuitry of the present disclosure may include a memory storing instructions or an algorithm which accounts for the loss of light due to the inefficiency in the first light pipe section and second light pipe section in determining the presence of a gas. The sensor is based on absorption of IR light at a particular wavelength by a gas the sensor is designed to detect the presence of. Because the light pipe does include some amount of inefficiency, the inefficiency is accounted for in an algorithm in order to prevent false positives.

### Example Proximity Sensors

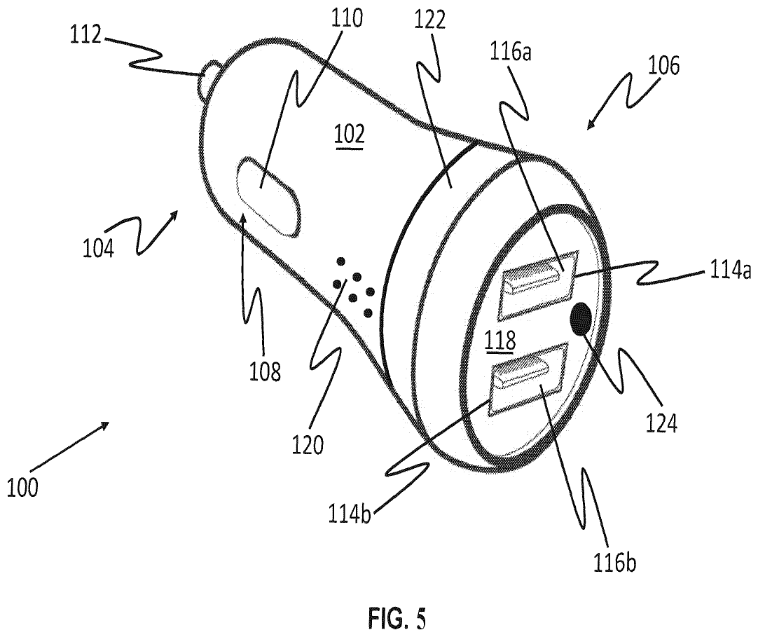

With reference to FIG. 5, the sensor 100 may take the general form of a power adapter which plugs in to an automotive cigarette lighter or similarly shaped power socket. The sensor may include a housing 102 formed from a plastic or similar material. For example, such a housing 102 may be formed using an injection molding process.

The housing 102 may have a distal end portion 104 and a proximal end portion 106. The distal end portion 104 may be shaped to fit in to, and connect with, the automotive cigarette lighter or power adapter. To that end, the distal end portion 104 may include interface surfaces 108. Specifically, the distal end may have a plurality of plates 110 which extend past an exterior surface of the housing 102 and abut the shell of the cigarette lighter or power adapter, when the sensor 100 is inserted, thereby providing at least partial connectivity to the electricity flowing to the cigarette lighter or power socket. The plates 110, or shell interface surfaces, may be leaf springs or may be spring loaded. The distal end portion 104 may further include a spring- loaded tip 112 which connects to a center contact of the cigarette lighter or power socket. The center contact may be positive and the shell negative or vice versa. Thus, the interface surfaces 108 allow the sensor 100 to draw electrical power from the cigarette lighter or power socket.

The proximal end portion 106 of the housing 102 may include openings 114a, 114b in which power ports 116a, 116b for charging cables may be placed. For example, these power ports 116a, 116b may be configured to accept a plug which complies with the universal serial bus standard. Alternatively, the power ports 116a, 116b may be configured to accept a plug of a different standard. A proximal end surface 118 of the housing 102 may be generally planar to make the placement of, and connection with, the power ports 116a, 116b easier. The proximal end portion 106 may further include an aperture 120 in the housing 102 for an audio driver's signal to pass. For example, the aperture 120 may be configured with the same shape as the perimeter of the audio driver, and the aperture 120 may have a screen across it. Alternatively, the aperture 120 may be a pattern of perforated holes placed in a location on the proximal end portion 106. The audio driver may be placed inside the housing 102 and the perforated holes allow the audio signal to pass through to the exterior of the housing 102.

The proximal end portion 106 may also include a visual indicator 122. For example, the visual indicator 122 may be a light emitting diode (LED), a small fluorescent lamp, or a small neon lamp. The visual indicator 122 may be any shape. In some embodiments the visual indicator 122 may be a ring around the housing. As will be discussed in more detail below regarding the operation of the sensor 100, the visual indicator 122 may operate in conjunction with the audio driver. Both may activate and emit in order to send an indication of a condition of operation of the sensor 100. Alternatively, there may be more than one visual indicator 122 on the proximal end portion 106. Each of the visual indicators 122 may signal a different operating condition of the sensor 100 when activated. Still further alternatively, one visual indicator 122 may have more than one color state. For example, the visual indicator 122 may indicate a first operational state when illuminated as a first color, and a second operational state when illuminated with a second color.

Additionally, the proximal end portion 106 may include a control surface 124 which may be actuated by a user. The control surface 124 may, for example, be a button or a switch. The control surface 124 may be electrically connected to the processor, so that when a user actuates the control surface 124, a signal is sent to the processor. Instructions on the memory may interpret this signal as an indicator to cancel the audio signal or visual indicator 122 activation, or both. This way, if the user intended for no smart devices to be present, the user is not forced to either unplug the sensor 100 or listen to the audio, look at the activated visual indicator 122, or both.

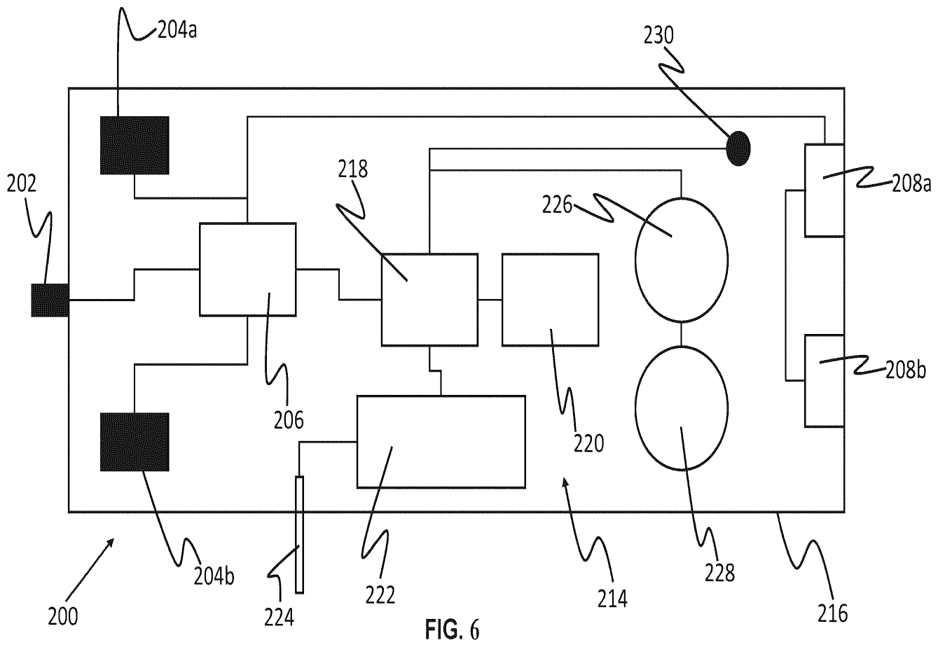

With reference to FIG. 6, the sensor 200, is an electrical device. Electrically, the interface surfaces, including the center contact interface surface 202, and the shell interface surfaces 204a, 204b, may be connected to a power supply 206. All the of the electrical components and circuitry with the exception of the interface surfaces 202, 204a, 204b, the power ports 208a, 208b, the audio driver 210, and the visual indicator 212 are located entirely in an interior 214 defined by the housing 216. The power supply 206 may regulate voltage from the 12 volts supplied by the car battery. The regulated voltage is required for proper operation of the sensitive electronic components in the sensor 200. The power supply 206 may be electrically connected to the remainder of the components or sub-circuits on the sensor 200.

The sensor 200 may include a processor. The processor 218 may be electrically connected to a memory 220 which may include instructions which are executed on the processor 218. The processor may be composed of multiple processor chips, with each chip performing separate assigned functions. For example, one processor chip may perform IEEE 802.11 encoding, and another processor chip may perform general device operations functions. The instructions which may be stored on the memory 220 and executed on the processor 218 are described in greater detail below with regard to the operation of the sensor 200. The processor 218 and the memory 220 may receive power from the power supply 206, either directly or indirectly. All the components of the sensor 200, including the processor 218 and memory 220 may be mounted on a printed circuit board (not shown), which may be configured to more easily fit all the components in the housing 216.

The processor 218 may be connected to a transceiver 222. The transceiver 222 may be configured to transmit and receiver IEEE 802.11 protocol configured transmissions in order to communicate with other devices wirelessly. These protocols may be stored on the memory 220, and executed on the processor 218 to operate the transceiver 222. The transceiver 222 may receive power from the power supply 206, as with the other components. The transceiver 222 may be shielded against interference from other components in the sensor 200. The transceiver 222 may include an antenna 224. The antenna 224 may be internal to the housing 216 or external of the housing 216. If the antenna 224 is external to the housing 216, the positioning of the antenna 224 may be adjustable.

The processor 218 may also be connected to a circuit or sub-circuit which includes the audio driver 226 and a circuit or sub-circuit which includes the visual indicator 228. Again, in some embodiments, there may be more than one visual indicator 228. The circuits or sub-circuits with the audio driver 226 and visual indicator 228 may be connected to the processor 218. In some embodiments, the audio driver 226 and the visual indicator 228 may be part of the same circuit or sub-circuit. These circuits or sub-circuits may include switches controlled by the processor 218. The switches control current flow to the audio driver 226 or to the visual indicator 228. When no current is flowing, no sound is emitted or visual indicators active. When current is flowing through the switch or switches, a signal is emitted from the audio driver 226 or the visual indicator 228 is active, or both.

In operation, power flows to the sensor 200 when the vehicle is started. If the vehicle is not running, typically the cigarette lighter or power socket has no power when the vehicle is not running in order to preserve the vehicle battery. Once the vehicle is running, power is routed to the cigarette lighter or power socket in to which the sensor 200 is plugged. The sensor 200 receives this power, and uses it for operation. The power is obtained by the interface surfaces 202, 204a, 204b and may have the voltage regulated by the power supply 206. The power may flow from the power supply 206, if one is present, to the various components.

Once the memory 220 and processor 218 receive instructions, the processor 218 may execute instructions which cause the IEEE 802.11 protocol transceiver 222 to begin searching for each of the smart devices stored on a list in the memory 220.

There may be further instructions which set parameters for this search. For example, the transceiver 222 may first check for any local area networks in range, if it finds at least one IEEE 802.11 protocol based local area network in range, the instructions may specify for the transceiver 222 to stop searching until the transceiver 222 no longer detects the local area network. The reason behind this instruction is that a smart device may only connect to one transceiver at a time. It may be very likely that the smart device is within range of both the local area network router and the transceiver 222 in the sensor 200. It is further very likely that the smart device is connected to the local area network and the transceiver 222 on the sensor 200 will only be able to connect with the smart device when the local area network is no longer in range of the smart device.

Further, the list of smart devices in the memory 220 itself may be a parameter. The transceiver 222 may only search for the smart devices on the list. Alternatively, the instructions stored on the memory 220 may cause the transceiver 222 to only search for only a portion of smart devices on the list. Alternatively, the memory 220 may include instructions which truncate the total time the transceiver 222 searches for the smart devices on the list. Each of these may speed the operation of the transceiver 222 and allow a user to become aware that a smart device is not in range of the sensor 200 sooner. This helps the user in various ways. For example, if the smart device is a smart phone, and the user left it at home and is driving away, the sensor 200 may alert the user to the fact sooner rather than later, making it easier for the user to return home and pick up the smart phone.

The transceiver 222 may search all the smart devices on the list. If the transceiver 222 does not connect to any of the smart devices on the list, the transceiver 222 may send a signal notifying the processor 218 of this condition. The instructions stored in the memory 220 may cause the processor 218 to send a signal to actuate the switch or switches which provide power to the audio driver 226 and visual indicator 228. This will alert the user to the condition that none of the smart devices on the list could be connected to the transceiver 222. The visual indicator 228 may light up only when no smart devices from the list could be connected. For example, the visual indicator 228 may be red when no smart devices could be connected.

Alternatively, the visual indicator 228 may use some, or all, of additional possible colors. For example, one embodiment of the sensor 200 may display a green light as a visual indicator 228 upon power up and when a device is connected. This same, or another, visual indicator may light yellow when the transceiver 222 is searching for smart devices. Finally, the same or another visual indicator 228 may light red when none of the smart devices on the list are connected. The memory 220 may include various instructions for execution on the processor 218 which cause various switches within the sensor 200 to be actuated to control the color changing of the visual indicator 228, or to turn various visual indicators 228 on and off in embodiments where more than one used.

The user may use a control surface 230 to send a signal to the processor 218 ending the audio signal, the activation of the visual indicator 228, or both. Alternatively, the control surface 230 may be used to reset the processor. Thus, a first or second press, when the first press ends the audio signal, the activation of the visual indicator 228, or both, of the control surface 230 may cause the instructions to reset the sensor 200, and the search to start over again. When the processor 218 has more than one processor chip, there may be a separate control surface for resetting each portion of the chip. For example, when the sensor 200 includes a main processor chip, and a WiFi® processor chip, the main processor chip may have a first control surface for resetting the main processor chip, and a second control surface for resetting the WiFi® processor chip. In addition, shutting off power to the sensor 200, for example, by turning off the vehicle, will cause the sensor 200 to reset, as well. The control surface 230 may be a button, a switch, a knob, or any mechanical or electrical, or combination thereof, component which will cause power to disconnect from the intended component.

Additionally, the memory 220 may store instructions which allow the sensor 200 to connect with a smart device for the purpose of changing various parameters of the sensor 200. These parameters may be stored in the memory 220 and may include the list of smart devices for which the sensor 200 is to search, the amount of time the sensor 200 is to spend searching for the smart devices on the list, and whether to toggle on or off a check for local area networks before performing a search for the smart devices stored in the list in the memory 220 of the sensor 200. The changes may be made by a user via a graphical user interface which is stored on the smart device. The graphical user interface may be part of an application stored on the smart device or may be created in real time by instructions stored on the memory 220 of the sensor 200.

The above description is given by way of example, and not limitation. Given the above disclosure, one skilled in the art could devise variations that are within the scope and spirit of the invention disclosed herein, including various ways of organizing visual indicators. Further, the various features of the embodiments disclosed herein can be used alone, or in varying combinations with each other and are not intended to be limited to the specific combination described herein. Thus, the scope of the claims is not to be limited by the illustrated embodiments.

### Example Embodiments

Below is a list of nonlimiting examples of implementations of the description above.

### Group 1

In a 1st Example, a sensor for detecting a proximity of a wireless connectable smart device using IEEE 802.11 protocols, comprising: a housing including a distal end portion and a proximal end portion; interface surfaces on the distal end portion to draw power from a power source; a power supply connected to the interface surfaces, the power supply regulating a voltage of the power; a transceiver using the IEEE 802.11 protocols located in the housing and electrically connected to the power supply; a processor in communication with the transceiver; a memory in communication with the processor, the memory having instructions stored on it for execution by the processor; and a notification system, wherein the instructions, executed by the processor, cause the transceiver to search for, and connect to, smart devices previously connected to the sensor using the IEEE 802.11 protocols, wherein if the transceiver fails to connect to any previously connected smart devices using the IEEE 802.11 protocols, the instructions cause the notification system to generate a notification.

In a 2nd Example, the sensor of Example 1, wherein the distal end portion is configured to be placed in an automotive cigarette lighter, and the proximal end portion including one or more power sockets for connecting one or more charging cables.

In a 3rd Example, the sensor of Example 1, wherein the transceiver is configured to connect to IEEE 802.11 protocol-capable smart devices within range of the transceiver.

In a 4th Example, the sensor of Example 1, wherein the notification system comprises a visual indicator configured to light a first color when power is flowing to the sensor and to light a second color when the notification is generated.

In a 5th Example, the sensor of Example 1, wherein the memory further includes a list of local area networks, and when the sensor is powered, the instructions, when executed by the processor, cause the transceiver to search for the local area networks and, if any are detected, to not perform the search for smart devices previously connected to the sensor using the IEEE 802.11 protocols.

In a 6th Example, the sensor of Example 4, wherein the instructions, when executed by the processor, allow a smart device connected using IEEE 802.11 protocols to be able to add and remove smart devices from a list of previously connected smart devices in the memory of the sensor and allow the smart device connected using IEEE 802.11 protocols to be able to add and remove local area networks from the list of local area networks.

In a 7th Example, the sensor of Example 1, wherein the notification system comprises an audio circuit in communication with the processor, the audio circuit including an audio driver for generating audible sounds, and wherein the notification comprises an audible sound.

In an 8th Example, a method for manufacturing a sensor for detecting a proximity of a smart device connectable to a transceiver using IEEE 802.11 protocols, comprising: forming a housing having a distal end portion configured to couple to a power source; providing a transceiver within an interior of the housing, the transceiver using IEEE 802.11 protocols to connected to IEEE 802.11 protocol capable smart devices within range of the transceiver; coupling a processor to the transceiver; coupling a memory to the processor, the memory having instructions stored on it for execution by the processor; and coupling a notification system to the processor; wherein, the instructions, when executed by the processor, cause the transceiver to search for, and connect to, smart devices previously connected to the sensor using the IEEE 802.11 protocols, wherein if the transceiver fails to connect to any previously connected smart devices using the IEEE 802.11 protocols, the instructions cause the notification system to generate a notification.

In a 9th Example, the method of Example 8, wherein the memory further includes a list of local area networks and wherein the instructions, when executed by the processor, are configured to cause the transceiver to search for the local area networks and, if any are detected, to not perform the search for smart devices previously connected to the sensor using the IEEE 802.11 protocols.

In a 10th Example, the method of Example 9, wherein the instructions, when executed by the processor, are configured to allow a smart device connected using IEEE 802.11 protocols to be able to add and remove smart devices from a list of previously connected smart devices in the memory of the sensor.

In a 11th Example, the method of Example 10, wherein the instructions, when executed by the processor, are configured to allow a smart device connected using IEEE 802.11 protocols to be able to add and remove local area networks from the list of local area networks.

In a 12th Example, the method of Example 8, wherein the notification system is configured to generate a visual indicator on an exterior of the housing, the visual indicator lighting a first color when power is flowing to the sensor, and changing to a second color when the notification is generated.

In a 13th Example, a sensor for detecting a proximity of a wireless connectable smart device using IEEE 802.11 protocols, comprising: a housing having a distal end portion configured to be coupled to a power source; interface surfaces on the distal end portion, the interface surfaces drawing power from the power source; a transceiver using the IEEE 802.11 protocols, the transceiver coupled to the interface surfaces, the transceiver configured to connect to IEEE 802.11 protocol capable smart devices within range of the transceiver; a processor coupled to the transceiver; a memory coupled to the processor, the memory having instructions stored on it for execution on the processor; and a notification system connected to the processor, the notification system configured to generate notifications; wherein, the instructions, when executed by the processor, are configured to cause the transceiver to search for, and connect to, smart devices previously connected to the sensor using the IEEE 802.11 protocols and, if the transceiver fails to connect to any previously connected smart devices using the IEEE 802.11 protocols, cause the notification system to generate a notification.

In a 14th Example, the sensor of Example 13, further comprising one or more charging ports located on a proximal end of the housing.

In a 15th Example, the sensor of Example 13, wherein the power source comprises a cigarette lighter.

In a 16th Example, the sensor of Example 13, wherein the memory further includes a list of local area networks, and when the sensor is powered, the instructions, when executed by the processor, are configured to cause the transceiver to search for the local area networks and, if any are detected, to not perform the search for smart devices previously connected to the sensor using the IEEE 802.11 protocols.

In a 17th Example, the sensor of Example 16, wherein the instructions, when executed by the processor, are configured to allow a smart device connected using IEEE 802.11 protocols to be able to add and remove smart devices from a list of previously connected smart devices in the memory of the sensor.

In an 18th Example, the sensor of Example 17, wherein the instructions, when executed by the processor, are configured to allow a smart device connected using IEEE 802.11 protocols to be able to add and remove local area networks from the list of local area networks.

In a 19th Example, the sensor of Example 13, wherein the notification system is configured to generate a visual indicator on an exterior of the housing, the visual indicator lighting a first color when power is flowing to the sensor, and changing to a second color when the notification is generated.

In a 20th Example, the sensor of Example 13, wherein the transceiver is configured to search for smart devices previously connected to the sensor using the IEEE 802.11 protocols for a maximum of 10 seconds.

### Group 2

In a 1st Example, a sensor for detecting the presence of one or more gasses, comprising: an IR lamp which generates light in the IR spectrum; a first light pipe section having a first end portion and a second end portion, the first end portion abutting the IR lamp and the first light pipe section passing the light through from the first end portion to the second end portion; a first sample tube section receiving light from the first light pipe section, the first sample tube section having a first interior containing a first sample, the first sample including a mix of gases from the local atmosphere, the IR spectrum light passing through the interior of the first sample tube section to a second end portion of the first sample tube section; a second light pipe section having an arcuate shape so that a first end and a second end of the second light pipe section face the same direction, the first end portion of the second light pipe section located adjacent to a second end portion of the first sample tube section, the second light pipe section receiving the light at the second end portion of the first sample tube section and transmitting the light from the first end portion to a second end portion; a second sample tube section with a first end portion and a second end portion, the first end portion of the second sample tube section located adjacent to the second end portion of the second light pipe section, the first end portion of the second sample tube section receiving the light from the second end of the second light pipe section, the second sample tube section having a second interior containing a second sample, the second sample including the mix of gases from the local atmosphere, the light passing through the interior of the second sample tube section to a second end portion of the second sample tube section; a first filter located at the second end portion of the second sample tube section, the first filter bandpassing only a band of IR spectrum light centered on a wavelength indicative of the presence of a gas in the mix of gases from the local atmosphere; and a first detector located behind to the first filter, the detector detecting the amount of IR spectrum light in the band of IR spectrum light.

### Other Considerations

The above description is given by way of example, and not limitation. Given the above disclosure, one skilled in the art could devise variations that are within the scope and spirit of the invention disclosed herein, including various ways of forming the syntax of the text or natural language commands. Further, the various features of the embodiments disclosed herein can be used alone, or in varying combinations with each other and are not intended to be limited to the specific combination described herein. Thus, the scope of the claims is not to be limited by the illustrated embodiments.

Any of the above-mentioned processors, and/or devices incorporating any of the above-mentioned processors, may be referred to herein as, for example, “computers,” “computer devices,” “computing devices,” “hardware computing devices,” “hardware processors,” “processing units,” and/or the like. Computing devices of the above-embodiments may generally (but not necessarily) be controlled and/or coordinated by operating system software, such as Mac OS, iOS, Android, Chrome OS, Windows OS (e.g., Windows XP, Windows Vista, Windows 7, Windows 8, Windows 10, Windows Server, etc.), Windows CE, Unix, Linux, SunOS, Solaris, Blackberry OS, VxWorks, or other suitable operating systems. In other embodiments, the computing devices may be controlled by a proprietary operating system. Conventional operating systems control and schedule computer processes for execution, perform memory management, provide file system, networking, I/O services, and provide a user interface functionality, such as a graphical user interface (“GUI”), among other things.

As described above, in various embodiments certain functionality may be accessible by a user through a web-based viewer (such as a web browser), or other suitable software program). In such implementations, the user interface may be generated by a server computing system and transmitted to a web browser of the user (e.g., running on the user's computing system). Alternatively, data (e.g., user interface data) necessary for generating the user interface may be provided by the server computing system to the browser, where the user interface may be generated (e.g., the user interface data may be executed by a browser accessing a web service and may be configured to render the user interfaces based on the user interface data). The user may then interact with the user interface through the web-browser. User interfaces of certain implementations may be accessible through one or more dedicated software applications. In certain embodiments, one or more of the computing devices and/or systems of the disclosure may include mobile computing devices, and user interfaces may be accessible through such mobile computing devices (for example, smartphones and/or tablets).

Many variations and modifications may be made to the above-described embodiments, the elements of which are to be understood as being among other acceptable examples. All such modifications and variations are intended to be included herein within the scope of this disclosure. The foregoing description details certain embodiments. It will be appreciated, however, that no matter how detailed the foregoing appears in text, the systems and methods can be practiced in many ways. As is also stated above, it should be noted that the use of particular terminology when describing certain features or aspects of the systems and methods should not be taken to imply that the terminology is being re-defined herein to be restricted to including any specific characteristics of the features or aspects of the systems and methods with which that terminology is associated.

Conditional language, such as, among others, “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

The term “substantially” when used in conjunction with the term “real-time” forms a phrase that will be readily understood by a person of ordinary skill in the art. For example, it is readily understood that such language will include speeds in which no or little delay or waiting is discernible, or where such delay is sufficiently short so as not to be disruptive, irritating, or otherwise vexing to a user.

Conjunctive language such as the phrase “at least one of X, Y, and Z,” or “at least one of X, Y, or Z,” unless specifically stated otherwise, is to be understood with the context as used in general to convey that an item, term, etc. may be either X, Y, or Z, or a combination thereof. For example, the term “or” is used in its inclusive sense (and not in its exclusive sense) so that when used, for example, to connect a list of elements, the term “or” means one, some, or all of the elements in the list. Thus, such conjunctive language is not generally intended to imply that certain embodiments require at least one of X, at least one of Y, and at least one of Z to each be present.

The term “a” as used herein should be given an inclusive rather than exclusive interpretation. For example, unless specifically noted, the term “a” should not be understood to mean “exactly one” or “one and only one”; instead, the term “a” means “one or more” or “at least one,” whether used in the claims or elsewhere in the specification and regardless of uses of quantifiers such as “at least one,” “one or more,” or “a plurality” elsewhere in the claims or specification.

The term “comprising” as used herein should be given an inclusive rather than exclusive interpretation. For example, a general purpose computer comprising one or more processors should not be interpreted as excluding other computer components, and may possibly include such components as memory, input/output devices, and/or network interfaces, among others.

While the above detailed description has shown, described, and pointed out novel features as applied to various embodiments, it may be understood that various omissions, substitutions, and changes in the form and details of the devices or processes illustrated may be made without departing from the spirit of the disclosure. As may be recognized, certain embodiments of the inventions described herein may be embodied within a form that does not provide all of the features and benefits set forth herein, as some features may be used or practiced separately from others. The scope of certain inventions disclosed herein is indicated by the appended claims rather than by the foregoing description. All changes which come within the meaning and range of equivalency of the claims are to be embraced within their scope.

What is claimed is:

1. A sensor for detecting a proximity of a wireless connectable smart device using IEEE 802.11 protocols, comprising:
a housing including a distal end portion and a proximal end portion;
interface surfaces on the distal end portion to draw power from a power source; a power supply connected to the interface surfaces, the power supply regulating a voltage of the power;
a transceiver using the IEEE 802.11 protocols located in the housing and electrically connected to the power supply;
a processor in communication with the transceiver;
a memory in communication with the processor, the memory having instructions stored on it for execution by the processor; and
a notification system,
wherein the instructions, executed by the processor, cause the transceiver to search for, and connect to, smart devices previously connected to the sensor using the IEEE 802.11 protocols, wherein if the transceiver fails to connect to any previously connected smart devices using the IEEE 802.11 protocols, the instructions cause the notification system to generate a notification.

2. The sensor of claim 1, wherein the distal end portion is configured to be placed in an automotive cigarette lighter, and the proximal end portion including one or more power sockets for connecting one or more charging cables.

3. The sensor of claim 1, wherein the transceiver is configured to connect to IEEE 802.11 protocol-capable smart devices within range of the transceiver.

4. The sensor of claim 1, wherein the notification system comprises a visual indicator configured to light a first color when power is flowing to the sensor and to light a second color when the notification is generated.

5. The sensor of claim 1, wherein the memory further includes a list of local area networks, and when the sensor is powered, the instructions, when executed by the processor, cause the transceiver to search for the local area networks and, if any are detected, to not perform the search for smart devices previously connected to the sensor using the IEEE 802.11 protocols.

6. The sensor of claim 4, wherein the instructions, when executed by the processor, allow a smart device connected using IEEE 802.11 protocols to be able to add and remove smart devices from a list of previously connected smart devices in the memory of the sensor and allow the smart device connected using IEEE 802.11 protocols to be able to add and remove local area networks from the list of local area networks.

7. The sensor of claim 1, wherein the notification system comprises an audio circuit in communication with the processor, the audio circuit including an audio driver for generating audible sounds, and wherein the notification comprises an audible sound.

8. A method for manufacturing a sensor for detecting a proximity of a smart device connectable to a transceiver using IEEE 802.11 protocols, comprising:
forming a housing having a distal end portion configured to couple to a power source;
providing a transceiver within an interior of the housing, the transceiver using IEEE 802.11 protocols to connected to IEEE 802.11 protocol capable smart devices within range of the transceiver;
coupling a processor to the transceiver;
coupling a memory to the processor, the memory having instructions stored on it for execution by the processor; and
coupling a notification system to the processor;
wherein, the instructions, when executed by the processor, cause the transceiver to search for, and connect to, smart devices previously connected to the sensor using the IEEE 802.11 protocols, wherein if the transceiver fails to connect to any previously connected smart devices using the IEEE 802.11 protocols, the instructions cause the notification system to generate a notification.

9. The method of claim 8, wherein the memory further includes a list of local area networks and wherein the instructions, when executed by the processor, are configured to cause the transceiver to search for the local area networks and, if any are detected, to not perform the search for smart devices previously connected to the sensor using the IEEE 802.11 protocols.

10. The method of claim 9, wherein the instructions, when executed by the processor, are configured to allow a smart device connected using IEEE 802.11 protocols to be able to add and remove smart devices from a list of previously connected smart devices in the memory of the sensor.

11. The method of claim 10, wherein the instructions, when executed by the processor, are configured to allow a smart device connected using IEEE 802.11 protocols to be able to add and remove local area networks from the list of local area networks.

12. The method of claim 8, wherein the notification system is configured to generate a visual indicator on an exterior of the housing, the visual indicator lighting a first color when power is flowing to the sensor, and changing to a second color when the notification is generated.

13. A sensor for detecting a proximity of a wireless connectable smart device using IEEE 802.11 protocols, comprising:
a housing having a distal end portion configured to be coupled to a power source;
interface surfaces on the distal end portion, the interface surfaces drawing power from the power source;
a transceiver using the IEEE 802.11 protocols, the transceiver coupled to the interface surfaces, the transceiver configured to connect to IEEE 802.11 protocol capable smart devices within range of the transceiver;
a processor coupled to the transceiver;
a memory coupled to the processor, the memory having instructions stored on it for execution on the processor; and
a notification system connected to the processor, the notification system configured to generate notifications;
wherein, the instructions, when executed by the processor, are configured to cause the transceiver to search for, and connect to, smart devices previously connected to the sensor using the IEEE 802.11 protocols and, if the transceiver fails to connect to any previously connected smart devices using the IEEE 802.11 protocols, cause the notification system to generate a notification.

14. The sensor of claim 13, further comprising one or more charging ports located on a proximal end of the housing.

15. The sensor of claim 13, wherein the power source comprises a cigarette lighter.

16. The sensor of claim 13, wherein the memory further includes a list of local area networks, and when the sensor is powered, the instructions, when executed by the processor, are configured to cause the transceiver to search for the local area networks and, if any are detected, to not perform the search for smart devices previously connected to the sensor using the IEEE 802.11 protocols.

17. The sensor of claim 16, wherein the instructions, when executed by the processor, are configured to allow a smart device connected using IEEE 802.11 protocols to be able to add and remove smart devices from a list of previously connected smart devices in the memory of the sensor.

18. The sensor of claim 17, wherein the instructions, when executed by the processor, are configured to allow a smart device connected using IEEE 802.11 protocols to be able to add and remove local area networks from the list of local area networks.

19. The sensor of claim 13, wherein the notification system is configured to generate a visual indicator on an exterior of the housing, the visual indicator lighting a first color when power is flowing to the sensor, and changing to a second color when the notification is generated.

20. The sensor of claim 13, wherein the transceiver is configured to search for smart devices previously connected to the sensor using the IEEE 802.11 protocols for a maximum of 10 seconds.
